(19) United States
(12) Patent Application Publication
(10) Pub. No.: US 2025/0234824 A1
(43) Pub. Date: Jul. 24, 2025

(54) MARKERS ASSOCIATED WITH SPONTANEOUS CHROMOSOME DOUBLING

(71) Applicant: Syngenta Crop Protection AG, Basel (CH)

(72) Inventors: Azhaguvel PERUMAL, Research Triangle Park, NC (US); Zhang YA, Beijing (CN); Becky Welsh Breitinger, Research Triangle Park, NC (US); Brent Delzer, Jaesville, WI (US); Rachel Louise Egger, Research Triangle Park, NC (US)

(73) Assignee: Syngenta Crop Protection AG, Basel (CH)

(21) Appl. No.: 18/839,801

(22) PCT Filed: Feb. 16, 2023
(86) PCT No.: PCT/US2023/062691

§ 371 Date: Aug. 20, 2024

(30) Foreign Application Priority Data
Feb. 23, 2022 (WO) PCT/CN2022/077351

(51) Int. Cl. A01H 1/04; A01H 1/08; A01H 5/10; A01H 6/46

(52) U.S. Cl. CPC A01H 1/04; A01H 1/08; A01H 5/10; A01H 6/4684

(57) ABSTRACT

Provided herein are methods for selecting and generating germplasm that displays spontaneous chromosome doubling when in a haploid state. The methods include isolating nucleic acids from a first maize plant or germplasm and detecting SCD-QTL1 associated with chromosome doubling. Further, the methods including selecting said first maize plant or germplasm, or a progeny of said first maize plant or germplasm, comprising SCD-QTL1 associated with spontaneous chromosome doubling.

### FIELD OF THE INVENTION

The presently disclosed subject matter relates generally to breeding and chromosome doubling in plants. More specifically, the subject matter relates to methods for obtaining and generating plants exhibiting spontaneous chromosome doubling when in a haploid state. Further, the methods herein relate to introgressing a locus of interest and a QTL associated with spontaneous chromosome doubling.

### SEQUENCE LISTING

This application is accompanied by a sequence listing entitled 82610_ST26.xml, created Dec. 15, 2022, which is approximately 24 kilobytes in size. This sequence listing is incorporated herein by reference in its entirety. This sequence listing is submitted herewith and is in compliance with 37 C.F.R. § 1.824(a) (2)-(6) and (b).

### BACKGROUND

Spontaneous chromosome doubling (“SCD”) is a rare trait in maize. Understanding its mechanism of action, and the loci associated with the trait, has been an area of academic research interest in recent years due to its potential value for doubled haploid breeding. Haploid plants are unable to produce fertile gametes during meiosis and must be doubled to obtain fertile pollen and egg cells. Thus, haploid maize plants without any intervention are expected to have entirely sterile pollen, except in the presence of SCD, which will allow for the correct meiotic reduction of chromosomes and subsequent formation of mature pollen. Various studies have identified 35 QTLs and SNPs associated with the trait in bi-allelic maize populations (Reviewed in Boerman, 2020). Several studies focused on mapping QTLs connected to SCD in Zea mays have identified loci on all maize chromosomes (Chaikam, 2019; Chaikam, 2020; Fuente, 2019; Ma, 2018; Molenaar, 2019; Ren, 2019; Vanous, 2019; Ren, 2017; Trampe, 2020; Wu, 2017; Yang, 2019). However, relatively few follow-up studies have been published. The exception is a strong QTL on chromosome 5 identified in several studies and containing the candidate gene BUBR1 identified in US20190106703. However, the QTL on chromosome 5 has not shown predictable penetrance across various germplasm in our hands. Therefore, there exists a need to identify a SCD QTL that affords better predictability and penetrance across germplasms.

### SUMMARY

Doubled haploid plants are of great interest, use, and advantage to plant breeders. Breeders cross inbred parent lines (also called elite lines), one acting as a male and one as a female, in order to form hybrid seed. The process of developing inbred parent lines which are substantially homozygous usually requires a plant to be selected and self-pollinated (selfed) for numerous generations to become nearly homozygous. This process is time consuming and expensive. To shorten the time to develop homozygous inbreds in maize, rice, wheat, barley, and other crops, breeders may opt to use a haploid inducer line to induce haploid seed production on a hybrid parent. Typically, obtaining doubled haploid plants involves obtaining a haploid plant as the product of a cross between a normal plant and a haploid-inducer plant, or by culturing haploid gametophytes into haploid sporophytes via microspore culture, anther culture, ovule culture, or ovary culture. Haploid plant sporophytes, absent some form of spontaneous chromosome doubling, are sterile if allowed to develop without further human intervention and contain only half the normal number of chromosomes for that plant species. See S. T. Chalyk, Properties of maternal haploid maize plants and potential application to maize breeding, EUPHYTICA 79:13-18 (1994), at 14, col. 2. For example, maize is considered a diploid organism comprising 20 chromosomes (i.e., two copies of each set of 10 distinct chromosomes). See, e.g., M. P. Maguire, Chromosome behavior at premeiotic mitosis in maize, J. HEREDITY 74:93-96 (1983), at Table 1. In comparison, a haploid maize plant contains only 10 chromosomes (i.e., one copy of each of the 10 chromosomes). To make a haploid plant fertile, the chromosome complement must be doubled, at least in the male and female reproductive lineages, if not the whole plant. Doubling of a plant's chromosomes is typically accomplished using a liquid mitotic spindle poison(s)—toxic chemicals sometimes used in treating cancers in humans or which chemicals may be antimicrotubule herbicides. See Y. Wan, et al., The use of antimicrotubule herbicides for the production of doubled haploid plants from anther-derived maize callus, THEOR. APPL. GENET. 81(2): 205-211 (1991). The chromosomes of the haploid plants are doubled by applying such a chromosome doubling agent (e.g., colchicine) to form doubled haploid homozygous inbred lines.

Without this doubling step, maize haploid plants are male infertile, that is, they lack the ability to make pollen. Here, we have identified a SCD QTL on maize chromosome 7 which, when present, indicates the maize plant is able to spontaneously double its haploid genome, at least in the florets, without the need to apply a chromosome doubling agent. Once this spontaneous doubling has occurred, the plant regains male fertility—i.e., it regains the ability to make pollen. We provide methods for selecting a first maize plant or germplasm displaying SCD when in a haploid state. Additionally provided are methods for obtaining a spontaneously doubled haploid plant that is male fertile. We also provide methods for crossing said first maize plant or germplasm with a second maize plant or germplasm to introgress a locus of interest, wherein the introgressed plant displays SCD in a haploid state. Further, we include methods for introgressing a locus of interest between a first and second maize plant, wherein a maize plant displaying the locus of interest and the SCD QTL is obtained. The locus of interest comprises a quantitative trait locus or allele associated with a trait selected from the group consisting of increased yield, increased water optimization, increased diseases resistance, increased drought tolerance, and increased herbicide resistance.

### BRIEF DESCRIPTION OF THE SEQUENCES IN THE SEQUENCE LISTING

SEQ ID NO: 1 is a forward primer used to amplify marker SM0077AQ in a TaqMan assay.

SEQ ID NO: 2 is a reverse primer used to amplify marker SM0077AQ in a TaqMan assay.

SEQ ID NO: 3 is a forward primer used to amplify marker SM11142 in a TaqMan assay.

SEQ ID NO: 4 is a forward primer used to amplify marker SM11142 in a TaqMan assay.

SEQ ID NO: 5 is a reverse primer used to amplify marker SM11142 in a TaqMan assay.

SEQ ID NO: 6 is the SM0077AQ marker target sequence.

SEQ ID NO: 7 is the SM11142 marker target sequence.

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. For example, the phrase “a cell” refers to one or more cells, and in some embodiments can refer to a tissue and/or an organ. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to all whole number values between 1 and 100 as well as whole numbers greater than 100.

Unless otherwise indicated, all numbers expressing quantities of ingredients, reaction conditions, and so forth used in the specification and claims are to be understood as being modified in all instances by the term “about.” The term “about,” as used herein when referring to a measurable value such as an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the discloses compositions, nucleic acids, polypeptides, etc. Accordingly, unless indicated to the contrary, the numerical parameters set forth in this specification and attached claims are approximations that can vary depending upon the desired properties sought to be obtained by the presently disclosed subject matter.

As used herein, “0% pollen fertility” or “male sterility” or “complete pollen sterility” in the context of this disclosure refers to 0% spontaneous chromosome doubling (SCD). More particularly, in this context, 0% pollen fertility refers to a haploid plant that cannot produce fertile pollen. In the present disclosure, because a haploid plant (1n genome) cannot be reduced to a 0.5n genome, the haploid plant also cannot make fertile pollen due to the inability to complete meiosis. Therefore, when referring to 0% pollen fertility in the present disclosure, the disclosure refers to 0% SCD. To produce fertile pollen from a haploid plant, the genome has to be doubled, either chemically or by SCD.

As used herein, the term “allele” refers to a variant or an alternative sequence form at a genetic locus. In diploids, a single allele is inherited by a progeny individual separately from each parent at each locus. The two alleles of a given locus present in a diploid organism occupy corresponding places on a pair of homologous chromosomes, although one of ordinary skill in the art understands that the alleles in any particular individual do not necessarily represent all of the alleles that are present in the species.

As used herein, the term “amplified” or “amplify” means the construction of multiple copies of a nucleic acid molecule or multiple copies complementary to the nucleic acid molecule using at least one of the nucleic acid molecules as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, PERSING et al., Ed., American Society for Microbiology, Washington, D.C. (1993). The product of amplification is termed an “amplicon.”

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D (e.g., AB, AC, AD, BC, BD, CD, ABC, ABD, and BCD). In some embodiments, one of more of the elements to which the “and/or” refers can also individually be present in single or multiple occurrences in the combinations(s) and/or subcombination(s).

As used herein, the phrase “associated with” refers to a recognizable and/or assayable relationship between two entities. For example, the phrase “associated with spontaneous chromosome doubling (SCD)” refers to a trait, locus, gene, allele, marker, phenotype, etc., or the expression thereof, the presence or absence of which can influence an extent and/or degree at which a plant is capable of SCD. As such, a marker is “associated with” a trait when it is linked to it and when the presence of the marker is an indicator of whether and/or to what extent the desired trait or trait form will occur in a plant/germplasm comprising the marker. Similarly, a marker is “associated with” an allele when it is linked to it and when the presence of the marker is an indicator of whether the allele is present in a plant/germplasm comprising the marker. For example, “a marker associated with SCD” refers to a marker whose presence or absence can be used to predict whether and/or to what extent a plant will display SCD.

As used herein, the term “backcrossing” is understood within the scope of the invention to refer to a process in which a hybrid progeny is repeatedly crossed back to one of the parents.

As used herein, the term “cDNA” refers to a single-stranded or a double-stranded DNA that is complementary to and derived from mRNA.

As used herein, the term “chromosome” is used herein as recognized in the art as meaning the self-replicating genetic structure in the cellular nucleus containing the cellular DNA and bearing the linear array of genes.

The term “comprising,” which is synonymous with “including,” “containing,” and “characterized by,” is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter.

With respect to the terms “comprising,” “consisting essentially of,” and “consisting of,” where one of these three terms is used herein, the presently disclosed and claimed subject matter can include in some embodiments the use of either of the other two terms. For example, if a subject matter relates in some embodiments to nucleic acids that encode polypeptides comprising amino acid sequences that are at least 95% identical to a SEQ ID NO: 2 or 3. It is understood that the disclosed subject matter thus also encompasses nucleic acids that encode polypeptides that in some embodiments consist essentially of amino acid sequences that are at least 95% identical to that SEQ ID NO: 2 or 3 as well as nucleic acids that encode polypeptides that in some embodiments consist of amino acid sequences that are at least 95% identical to that SEQ ID NO: 2 or 3. Similarly, it is also understood that in some embodiments the methods for the disclosed subject matter comprise the steps that are disclosed herein, in some embodiments the methods for the presently disclosed subject matter consist essentially of the steps that are disclosed, and in some embodiments the methods for the presently disclosed subject matter consist of the steps that are disclosed herein.

As used herein, the term “de novo haploid induction” refers to the triggering of haploid induction by the introduction of a spontaneous-haploid inducing agent. Such introduction can be achieved by topical spray, hand-pollination, mutagenesis, or transgenic methods. The terms “de novo haploid induction,” “de novo HI,” and “haploid induction de novo” are used interchangeably throughout this specification.

As used herein, the term “elite line” or “inbred line” refers to any line that has resulted from breeding and selection for superior agronomic performance. An elite line has stable genetics, i.e., it is reasonably or nearly isogenic across its genome. Said another way, an elite line is reasonably or nearly homozygous for all alleles in its genome.

As used herein, the term “expression” when used with reference to a polynucleotide, such as a gene, ORF or portion thereof, or a transgene in plants, refers to the process of converting genetic information encoded in a gene into RNA (e.g., mRNA, rRNA, tRNA, or snRNA) through “transcription” of the gene (i.e., via the enzymatic action of an RNA polymerase), and into protein where applicable (e.g. if a gene encodes a protein), through “translation” of mRNA. Gene expression can be regulated at many stages in the process. For example, in the case of antisense or dsRNA constructs, respectively, expression may refer to the transcription of the antisense RNA only or the dsRNA only. In embodiments, “expression” refers to the transcription and stable accumulation of sense (mRNA) or functional RNA. “Expression” may also refer to the production of protein.

As used herein, the term “gene” refers to a hereditary unit including a sequence of DNA that occupies a specific location on a chromosome and that contains the genetic instruction for a particular characteristic or trait in an organism.

As used herein, the term “genotype” refers to the genetic constitution of a cell or organism. An individual's “genotype for a set of genetic markers” includes the specific alleles, for one or more genetic marker loci, present in the individual. As is known in the art, a genotype can relate to a single locus or to multiple loci, whether the loci are related or unrelated and/or are linked or unlinked. In some embodiments, an individual's genotype relates to one or more genes that are related in that the one or more of the genes are involved in the expression of a phenotype of interest (e.g., a quantitative trait as defined herein). Thus, in some embodiments a genotype comprises a sum of one or more alleles present within an individual at one or more genetic loci of a quantitative trait. In some embodiments, a genotype is expressed in terms of a haplotype (defined herein below).

A “genetic map” is a description of genetic linkage relationships among loci on one or more chromosomes within a given species, generally depicted in a diagrammatic or tabular form.

As used herein, the term “germplasm” refers to the totality of the genotypes of a population or another group of individuals (e.g., a species). The term “germplasm” can also refer to plant material; e.g., a group of plants that act as a repository for various alleles. The phrase “adapted germplasm” refers to plant materials of proven genetic superiority; e.g., for a given environment or geo-graphical area, while the phrases “non-adapted germplasm”, “raw germplasm”, and “exotic germplasm” refer to plant materials of unknown or unproven genetic value; e.g., for a given environment or geographical area; as such, the phrase “non-adapted germplasm” refers in some embodiments to plant materials that are not part of an established breeding population and that do not have a known relationship to a member of the established breeding population.

As used herein, a plant referred to as “haploid” has a single set (genome) of chromosomes and the reduced number of chromosomes (In) in the haploid plant is equal to that of the gamete. As used herein, a plant referred to as “doubled haploid” is developed by doubling the haploid set of chromosomes (from 1n to 2n). A plant or seed that is obtained from a doubled haploid plant that is selfed to any number of generations may still be identified as a doubled haploid plant. A doubled haploid plant is considered a homozygous plant. A plant is considered to be doubled haploid if it is fertile, even if the entire vegetative part of the plant does not consist of the cells with the doubled set of chromosomes; that is, a plant will be considered doubled haploid if it contains viable gametes, even if it is chimeric.

As used herein, the term “heterologous” when used in reference to a gene or nucleic acid refers to a gene encoding a factor that is not in its natural environment (i.e., has been altered by the hand of man). For example, a heterologous gene may include a gene from one species introduced into another species. A heterologous gene may also include a gene native to an organism that has been altered in some way (e.g., mutated, added in multiple copies, linked to a non-native promoter or enhancer polynucleotide, etc.). Heterologous genes further may comprise plant gene polynucleotides that comprise cDNA forms of a plant gene; the cDNAs may be expressed in either a sense (to produce mRNA) or anti-sense orientation (to produce an anti-sense RNA transcript that is complementary to the mRNA transcript). In one aspect of the invention, heterologous genes are distinguished from endogenous plant genes in that the heterologous gene polynucleotide are typically joined to polynucleotides comprising regulatory elements such as promoters that are not found naturally associated with the gene for the protein encoded by the heterologous gene or with plant gene polynucleotide in the chromosome, or are associated with portions of the chromosome not found in nature (e.g., genes expressed in loci where the gene is not normally expressed). Further, in embodiments, a “heterologous” polynucleotide is a polynucleotide not naturally associated with a host cell into which it is introduced, including non-naturally occurring multiple copies of a naturally occurring polynucleotide.

As used herein, the term “heterozygous” means a genetic condition existing when different alleles reside at corresponding loci on homologous chromosomes.

As used herein, the term “homozygous” means a genetic condition existing when identical alleles reside at corresponding loci on homologous chromosomes.

As used herein, “HMF score” or “Haploid Male Fertility score” refers to the number of days a plant sheds pollen. The method used herein measures each individual plant. Plants are checked for fertile pollen shed everyday over the course of the pollen shedding period (7-14 days in the field), and each plant is assigned a number equal to the number of days it shed pollen (e.g., a plant assigned number 5 equals a plant shedding pollen for 5 days). A sterile plant is scored as a zero, meaning there were zero days it shed pollen. In the absence of other interventions, any haploid plant with a score greater than zero is a haploid plant with some level of SCD.

As used herein, the term “isolated,” when used in the context of the nucleic acid molecules or polynucleotides of the present invention, refers to a polynucleotide that is identified within and isolated/separated from its chromosomal polynucleotide context within the respective source organism. An isolated nucleic acid or polynucleotide is not a nucleic acid as it occurs in its natural context, if it indeed has a naturally occurring counterpart. In contrast, non-isolated nucleic acids are nucleic acids such as DNA and RNA, which are found in the state they exist in nature. For example, a given polynucleotide (e.g., a gene) is found on the host cell chromosome in proximity to neighboring genes. The isolated nucleic acid molecule may be present in single-stranded or double-stranded form. Alternatively, it may contain both the sense and antisense strands (i.e., the nucleic acid molecule may be double-stranded). In a preferred embodiment, the nucleic acid molecules of the present invention are understood to be isolated.

As used herein, the term “locus” refers to a position (e.g., of a gene, a genetic marker, or the like) on a chromosome of a given species.

As used herein, “maternal haploid inducer” refers to a line that produces pollen and, when crossed as a male, results in the gynogenic development of haploid seeds. A “paternal haploid inducer” refers to a line that when used as a female in a cross, results in androgenic development of haploid seeds. A haploid inducer plant can use either of these maternal or paternal mechanisms to derive haploids.

As used herein, the term “human-induced mutation” refers to any mutation that occurs as a result of either direct or indirect human action. This term includes, but is not limited to, mutations obtained by any method of targeted mutagenesis.

As used herein, the term “hybrid” refers to offspring produced by crossing two genetically dissimilar parent plants. The resulting progeny of this cross are a “bi-parental” population.

As used herein, “logarithm of the odds” or “LOD score” refers to the statistical likelihood that two genes are in proximity on a chromosome.

As used herein, the terms “marker probe” and “probe” refer to a nucleotide sequence or nucleic acid molecule that can be used to detect the presence or absence of a sequence within a larger sequence, e.g., a nucleic acid probe that is complementary to all of or a portion of the marker or marker locus, through nucleic acid hybridization. Marker probes comprising about 8, 10, 15, 20, 30, 40, 50, 60, 70, 80, 90, 100 or more contiguous nucleotides can be used for nucleic acid hybridization.

As used herein, the term “molecular marker” can be used to refer to a genetic marker, as defined above, or an encoded product thereof (e.g., a protein) used as a point of reference when identifying the presence/absence of a SCD or other locus of interest. A molecular marker can be derived from genomic nucleotide sequences or from expressed nucleotide sequences (e.g., from an RNA, a cDNA, etc.). The term also refers to nucleotide sequences complementary to or flanking the marker sequences, such as nucleotide sequences used as probes and/or primers capable of amplifying the marker sequence. Nucleotide sequences are “complementary” when they specifically hybridize in solution (e.g., according to Watson-Crick base pairing rules). This term also refers to the genetic markers that indicate a trait by the absence of the nucleotide sequences complementary to or flanking the marker sequences, such as nucleotide sequences used as probes and/or primers capable of amplifying the marker sequence.

As used herein, the terms “nucleotide sequence,” “polynucleotide,” “nucleic acid sequence,” “nucleic acid molecule,” and “nucleic acid fragment” refer to a polymer of RNA or DNA that is single- or double-stranded, optionally containing synthetic, non-natural, and/or altered nucleotide bases. A “nucleotide” is a monomeric unit from which DNA or RNA polymers are constructed and consists of a purine or pyrimidine base, a pentose, and a phosphoric acid group. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The term “identity” or “identical” in the context of two nucleic acid or amino acid sequences, refers to the percentage of identical nucleotides or amino acids in a linear polynucleotide or amino acid sequence of a reference (“query”) sequence (or its complementary strand) as compared to a test (“subject”) sequence when the two sequences are globally aligned. Unless otherwise stated, sequence identity as used herein refers to the value obtained using the Needleman and Wunsch algorithm ((1970) J. Mol. Biol. 48:443-453) implemented in the EMBOSS Needle alignment tool using default matrix files EBLOSUM62 for protein with default parameters (Gap Open=10, Gap Extend=0.5, End Gap Penalty=False, End Gap Open=10, End Gap Extend=0.5) or DNAfull for nucleic acids with default parameters (Gap Open=10, Gap Extend=0.5, End Gap Penalty=False, End Gap Open=10, End Gap Extend=0.5); or any equivalent program thereof. EMBOSS Needle is available, e.g., from EMBL-EBI such as at the following website: ebi.ac.uk/Tools/psa/emboss_needle/and as described in the following publication: “The EMBL-EBI search and sequence analysis tools APIs in 2019.” Madeira et al. Nucleic Acids Research, June 2019, 47(W1): W636-W641. The term “equivalent program” as used herein refers to any sequence comparison program that, for any two sequences in question, generates an alignment having identical nucleotide or amino acid residue matches and an identical percent sequence identity when compared to the corresponding alignment generated by EMBOSS Needle. In some embodiments, substantially identical nucleic acid or amino acid sequences may perform substantially the same function.

As used herein, the terms “introgression”, “introgressed” and “introgressing” refer to both a natural and artificial process whereby genomic regions of one species, variety or cultivar are moved into the genome of another species, variety or cultivar, by crossing those species. The process may optionally be completed by backcrossing to the recurrent parent.

The term “open reading frame” (ORF) refers to a nucleic acid sequence that encodes a polypeptide. In some embodiments, an ORF comprises a translation initiation codon, a translation termination (i.e., stop) codon, and the nucleic acid sequence there between that encodes the amino acids present in the polypeptide. The terms “initiation codon” and “termination codon” refer to a unit of three adjacent nucleotides (i.e., a codon) in a coding sequence that specifies initiation and chain termination, respectively, of protein synthesis (mRNA translation).

As used herein, the terms “phenotype,” “phenotypic trait” or “trait” refer to one or more traits of a plant or plant cell. The phenotype can be observable to the naked eye, or by any other means of evaluation known in the art, e.g., microscopy, biochemical analysis, or an electromechanical assay. In some cases, a phenotype is directly controlled by a single gene or genetic locus (i.e., corresponds to a “single gene trait”). For example, in the case of a haploid plant evaluated for SCD, phenotype can refer to fertile pollen shed and/or a seed obtained via pollination with that pollen. In other cases, a phenotype is the result of interactions among several genes, which in some embodiments also results from an interaction of the plant and/or plant cell with its environment.

As used herein, the term “plant” can refer to a whole plant, any part thereof, or a cell or tissue culture derived from a plant. Thus, the term “plant” can refer to any of: whole plants, plant components or organs (e.g., leaves, stems, roots, etc.), plant tissues, seeds and/or plant cells, unless otherwise specified.

A plant cell is a cell of a plant, taken from a plant, or derived through culture from a cell taken from a plant. Thus, the term “plant cell” includes without limitation cells within seeds, suspension cultures, embryos, meristematic regions, callus tissue, leaves, shoots, gametophytes, sporophytes, pollen, and microspores. The phrase “plant part” refers to a part of a plant, including single cells and cell tissues such as plant cells that are intact in plants, cell clumps, and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as scions, rootstocks, protoplasts, calli, and the like.

As used herein, the term “population” means a genetically heterogeneous collection of plants sharing a common genetic derivation.

As used herein, the term “primer” refers to an oligonucleotide which is capable of annealing to a nucleic acid target (in some embodiments, annealing specifically to a nucleic acid target) allowing a DNA polymerase and/or reverse transcriptase to attach thereto, thereby serving as a point of initiation of DNA synthesis when placed under conditions in which synthesis of a primer extension product is induced (e.g., in the presence of nucleotides and an agent for polymerization such as DNA polymerase and at a suitable temperature and pH). In some embodiments, one or more pluralities of primers are employed to amplify plant nucleic acids (e.g., using the polymerase chain reaction; PCR).

As used herein, the term “probe” refers to a nucleic acid (e.g., a single stranded nucleic acid or a strand of a double stranded or higher order nucleic acid, or a subsequence thereof) that can form a hydrogen-bonded duplex with a complementary sequence in a target nucleic acid sequence. Typically, a probe is of sufficient length to form a stable and sequence-specific duplex molecule with its complement, and as such can be employed in some embodiments to detect a sequence of interest present in a plurality of nucleic acids.

As used herein, the terms “progeny” and “progeny plant” refer to a plant generated from a vegetative or sexual reproduction from one or more parent plants. In haploid induction the seed on the female parent is haploid, thus not a progeny of the inducing haploid line. The progeny of the haploid seed is not the only desired progeny. There is also the HI seed and subsequent plant and seed progeny of the haploid inducing plant. Both the haploid seed and the HI seed can be progeny. A progeny plant can be obtained by cloning or selfing a single parent plant, or by crossing two or more parental plants. For instance, a progeny plant can be obtained by cloning or selfing of a parent plant or by crossing two parental plants and include selfings as well as the F1 or F2 or still further generations. An F1 is a first-generation progeny produced from parents at least one of which is used for the first time as donor of a trait, while progeny of second generation (F2) or subsequent generations (F3, F4, and the like) are specimens produced from selfings, intercrosses, backcrosses, and/or other crosses of F1s, F2s, and the like. An F1 can thus be (and in some embodiments is) a hybrid resulting from a cross between two true breeding parents (i.e., parents that are true-breeding are each homozygous for a trait of interest or an allele thereof), while an F2 can be (and in some embodiments is) a progeny resulting from self-pollination of the F1 hybrids.

As used herein, “F1-H generation” refers to progeny produced by first crossing an inbred containing the SCD trait with an inbred line absent the SCD trait (F1). The progeny from said first cross was then crossed by a maternal haploid inducer line, producing the F1-H generation. As used herein, “F2-H generation” refers to progeny produced by said F1 plants above selfing to produce F2, which was then crossed by a haploid inducer.

As used herein, “QTL penetrance” refers to the presence of an associated phenotype with a QTL locus.

As used herein, “quantitative trait locus” or “quantitative trait loci” or “QTL” refers to a locus associated with a particular trait.

As used herein, “R1-nj” refers to the R1-Navajo anthocyanin marker. It is a marker well-known in the art used as a visual marker for distinguishing haploids from diploids (or aneuploids).

As used herein, the phrase “recombination” refers to an exchange of DNA fragments between two DNA molecules or chromatids of paired chromosomes (a “crossover”) over in a region of similar or identical nucleotide sequences. A “recombination event” is herein understood to refer in some embodiments to a meiotic crossover.

As used herein, the term “reference sequence” refers to a defined nucleotide sequence used as a basis for nucleotide sequence comparison.

As used herein, the term “regenerate,” and grammatical variants thereof, refers to the production of a plant from tissue culture.

As used herein, “spontaneous chromosome doubling” (“SCD”) or “spontaneous haploid genome doubling” or “haploid male fertility” or “spontaneous genome doubling” are used interchangeably to describe the doubling of haploid genomes without any intervention. SCD allows for the correct meiotic reduction of chromosomes and subsequent formation of mature pollen. In the present disclosure, SCD was calculated by dividing the number of fertile haploid plants/by the total number of plants.

As used herein, “spontaneously doubled haploid plant” refers to a plant whose florets have undergone spontaneous doubling. Other tissues of a spontaneously doubled haploid plant may retain their haploid state (e.g., root, leaf, stem).

As used herein, “SCD-QTL1” refers to the presently identified QTL on chromosome 7. This QTL is defined by the region of chromosome 7 flanked by two markers, SM0077AQ and SM11142. These markers correspond to their physical locations in the B73_v4 reference genome.

As used herein, the phrase “stringent hybridization conditions” refers to conditions under which a polynucleotide hybridizes to its target subsequence, typically in a complex mixture of nucleic acids, but to essentially no other sequences. Stringent conditions are sequence-dependent and can be different under different circumstances.

Longer sequences typically hybridize specifically at higher temperatures. An extensive guide to the hybridization of nucleic acids is found in Sambrook & Russell, 2001. Generally, stringent conditions are selected to be about 5-10° C. lower than the thermal melting point (Tm) for the specific sequence at a defined ionic strength pH. The Tm is the temperature (under defined ionic strength, pH, and nucleic acid concentration) at which 50% of the probes complementary to the target hybridize to the target sequence at equilibrium (as the target sequences are present in excess, at Tm, 50% of the probes are occupied at equilibrium). Exemplary stringent conditions are those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides).

Stringent conditions can also be achieved with the addition of destabilizing agents such as formamide. Additional exemplary stringent hybridization conditions include 50% formamide, 5×SSC, and 1% SDS incubating at 42° C.; or SSC, 1% SDS, incubating at 65° C.; with one or more washes in 0.2×SSC and 0.1% SDS at 65° C. For PCR, a temperature of about 36° C. is typical for low stringency amplification, although annealing temperatures can vary between about 32° C. and 48° C. (or higher) depending on primer length. Additional guidelines for determining hybridization parameters are provided in numerous references (see e.g., Ausubel et al., 1999).

As used herein, the term “trait” refers to a phenotype of interest, a gene that contributes to a phenotype of interest, as well as a nucleic acid sequence associated with a gene that contributes to a phenotype of interest.

As used herein, the term “transgene” refers to a nucleic acid molecule introduced into an organism or one or more of its ancestors by some form of artificial transfer technique. The artificial transfer technique thus creates a “transgenic organism” or a “transgenic cell.” It is understood that the artificial transfer technique can occur in an ancestor organism (or a cell therein and/or that can develop into the ancestor organism) and yet any progeny individual that has the artificially transferred nucleic acid molecule or a fragment thereof is still considered transgenic even if one or more natural and/or assisted breedings result in the artificially transferred nucleic acid molecule being present in the progeny individual.

As used herein, the term “targeted mutagenesis” or “mutagenesis strategy” refers to any method of mutagenesis that results in the intentional mutagenesis of a chosen gene. Targeted mutagenesis includes the methods CRISPR, TILLING, TALEN, and other methods not yet discovered but which may be used to achieve the same outcome.

As used herein, haploid induction rate (“HIR”) means the number of surviving haploid kernels over the total number of kernels after an ear is pollinated with haploid inducer pollen.

### DETAILED DESCRIPTION

The present disclosure relates to, among other things, spontaneous doubling haploid plants. In one embodiment, the disclosure is a method of selecting a first maize plant or germplasm that displays spontaneous chromosome doubling (“SCD”) when in a haploid state. The method comprises isolating nucleic acids from the first maize plant or germplasm and detecting SCD-QTL1 comprising SEQ ID NOs: 6 and 7 that is associated with spontaneous chromosome doubling. Further, the method comprises selecting said first maize plant or germplasm, or selecting a progeny of said first maize plant or germplasm, comprising SCD-QTL1 associated with spontaneous chromosome doubling. In another embodiment, the above method further comprises crossing said selected first maize plant or germplasm with a second maize plant or germplasm. The introgressed maize plant or germplasm displays increased spontaneous chromosome doubling (“SCD”) when in a haploid state. The SCD-QTL1 is detected using a composition comprising a detectable label.

In another embodiment, the present disclosure includes a method of obtaining a spontaneously doubled haploid plant. The method comprises obtaining a first maize plant comprising SCD-QTL1 comprising SEQ ID NOs: 6 and 7 that is associated with spontaneous chromosome doubling, crossing the first maize plant with a second maize plant, obtaining progeny from said cross, selecting haploid progeny therefrom, and allowing the haploid progeny to undergo spontaneous chromosome doubling. The spontaneously doubled haploid plant may comprise reproductive tissues competent to produce fertile gametes. The spontaneously doubled haploid plant is male fertile.

In another embodiment, the first maize plant is a haploid inducer maize plant. The first maize plant comprises a mutation in CENH3 or IG1 (see, e.g., U.S. Pat. No. 8,618,354 and PCT Application Publication No. WO2011/044132, incorporated herein by reference in their entireties). In another embodiment, the second maize plant is a haploid inducer maize plant. The second maize plant comprises a mutation in ZmMATL (see, e.g., U.S. Pat. Nos. 9,677,082, 10,448,588, and PCT Application Publication No. WO2017/087682, incorporated herein by reference in their entireties).

In another embodiment, the disclosure includes a method of introgressing a locus of interest. The method comprises first obtaining a first maize plant comprising SCD-QTL1 comprising SEQ ID NOs: 6 and 7 that is associated with spontaneous chromosome doubling. The method then includes crossing the first maize plant with a second maize plant comprising the locus of interest and obtaining progeny from the cross of the first maize plant and the second maize plant. Further, the method optionally includes backcrossing the progeny obtained from crossing the first and second maize plants from above with the first maize plant or the second maize plant. The method further includes screening the progeny of the previously mentioned crosses for presence of the locus of interest and the SCD QTL and selecting a progeny at least heterozygous for the locus of interest and at least heterozygous for the SCD QTL. In an embodiment, the progeny screening for presence of the locus of interest and the SCD-QTL is screened by genotype. In another embodiment, the progeny screening for presence of the locus of interest and the SCD-QTL is screened by phenotype. Further, a maize plant is produced by the method, and said maize plant belongs to the non-stiff stalk heterotic group, the stiff stalk heterotic group, or the tropical heterotic group. A maize progeny plant is produced from the maize plant. The maize plant is a hybrid maize plant or an elite maize plant.

In another embodiment, the locus of interest of the method of introgressing the locus of interest comprises a quantitative trait locus that is associated with a trait selected from the group consisting of increased yield, increased water optimization, increased disease resistance, increased drought tolerance, and increased herbicide resistance. Further, the locus of interest comprises a transgene.

### EXAMPLES

### Example 1. Screening for SCD in Syngenta Maize Germplasm

To better understand the SCD trait, we screened 20 lines reported to have observed SCD by maize breeders. This screen was performed by crossing proprietary inbred lines with a haploid inducer, isolating haploids using a genetic color marker, and growing the regenerated haploid plants in the absence of any chemical doubling agents under typical greenhouse conditions. Under these conditions, typical maize haploids will be 100% male sterile and shed no pollen (0% pollen fertility is defined as 0% SCD). Plants were evaluated for pollen fertility on a daily basis during the period of pollen shed and any pollen shed (i.e. released into the air) from the tassels was presumed to be fertile. Male sterile maize plants typically fail to have any anthers emerge from the tassel and no pollen shed is observed in these cases. However, four of these lines (SYN004, SYN007, SYN013, and K22) exhibited SCD at a rate greater than 15%. For the remaining 16 lines tested, the average SCD was 5% (See Table 1). A subset of lines, including some of the top performers, were also grown under field conditions to confirm the greenhouse observations (See Table 1). Four of the top performing lines were advanced to generate QTL mapping populations for the SCD trait.

TABLE 1
Phenotypic scores from 20 lines screened for SCD
trait in greenhouse and field environments

| | SCD rate Greenhouse (Fertile | | SCD rate Field (Fertile | |
|---|---|---|---|---|
| Line Name | Haploids/Total) | | Haploids/Total) | |
| Zheng58 | 0% | (0/24) | 5% | (1/20) |
| SYN001 | 5% | (1/22) | No Data | |
| SYN002 | 15% | (2/13) | No Data | |
| SYN003 | 8% | (2/25) | No Data | |
| SYN004 | 100% | (23/23) | 100% | (21/21) |
| SYN005 | 12% | (3/25) | No Data | |
| SYN006 | 4% | (1/24) | No Data | |
| SYN007 | 96% | (24/25) | 85% | (18/21) |
| SYN008 | 4% | (1/24) | No Data | |
| SYN009 | 10% | (2/21) | No Data | |
| SYN010 | 8% | (2/24) | No Data | |
| SYN011 | 0% | (0/15) | No Data | |
| K22 | 72% | (13/18) | 68% | (13/19) |
| SYN012 | 0% | (0/24) | No Data | |
| SYN013 | 63% | (15/24) | No Data | |
| NP2222 | 10% | (2/21) | 10% | (2/20) |

| | | | |
|---|---|---|---|
| SYN014 | 0% | (0/20) | No Data |
| SYN015 | 5% | (1/20) | No Data |
| SYN016 | 4% | (1/23) | No Data |
| SYN017 | 0% | (0/23) | No Data |

### Example 2. QTL Identification and Validation

### QTL Identification in Bi-Parental F1-H Population

To map the SCD trait, bi-parental populations were generated by crossing an inbred line with the SCD trait (examples: SYN004 and SYN007) with inbred lines that did not exhibit the high rate of SCD phenotype (examples: NP2222 and SYN006). The F1 progeny of that cross (presumed to be heterozygous for any SCD-associated loci) was then crossed by a maternal haploid inducer line, RWK, and seed sorted for haploids using a selectable marker (see, for example, Iowa State University's website: www.doubledhaploid.biotech.iastate.edu/haploid-selection) (the “Hohenheim method”), producing an F1-H generation. Haploid seeds were sown in a sunlight greenhouse. Individual plants were phenotyped by counting the number of days of sequential pollen shed (HMF Score) and by assessing kernels produced from a self-pollination to confirm that the observed pollen was viable. Individual plants were genotyped using an Affymetrix Axiom chip array (see for example thermofisher.com/us/en/home/life-science/microarray-analysis/agrigenomics-solutions-microarrays-gbs/axiom-genotyping-solution-agrigenomics), and the array results correlated with the phenotypic results to determine regions of the genome associated with the doubling trait. For two of the SCD inbred lines, no meaningful QTL were identified in the association mapping. For the remaining lines (SYN004 and SYN007), a total of four QTL with LOD scores>5 were considered significant, especially when similar loci appeared in multiple F1-H populations (Table 2).

TABLE 2
QTL identified from F1-H populations with relative LOD scores and the explained
variance for the trait associated with that region. QTL with overlapping intervals are
grouped together in the table when they appeared in independent F1-H populations. The
physical locations on their respective chromosomes are in reference to the
B73_v4 maize genome, which is publicly available at maizegdb.org
(maizegdb.org/genome/assembly/Zm-B73-REFERENCE-GRAMENE-4.0).

| | | B73_v4 | B73_v4 | | |
|---|---|---|---|---|---|
| | | Physical Left | Physical Right | | |
| F1 -H Population | | Flanking | Flanking | LOD | Explained |
| Parent Lines | Chromosome | Marker | Marker | Avg | Variance |
| NP2222-SYN007 | 2 | 3,825,522 | 3,876,621 | 16.02 | 41.52 |
| SYN007- | 7 | 122,017,320 | 122,293,652 | 5.28 | 27.37 |
| KDJ4350 | | | | | |
| SYN006 - | 7 | 122,293,652 | 122,333,069 | 5.67 | 10.75 |
| SYN004 | | | | | |
| SYN007- | 7 | 130,298,062 | 130,348,021 | 5.26 | 25.53 |
| KDJ4350 | | | | | |
| SYN004 NP2222 | 7 | 130,393,825 | 129,448,023 | 26.02 | 40.77 |
| SYN007- | 10 | 143,284,946 | 143,503,768 | 5.63 | 29.65 |
| KDJ4350 | | | | | |
| SYN007- | 10 | 148,400,298 | 148,409,257 | 6.91 | 49.35 |
| KDJ4350 | | | | | |

### QTL Validation in Bi-Parental F2-H Population

Validation of these four QTL were conducted in the following generation. The F1 plants from the bi-parental population were selfed to produce an F2 which was then crossed by the haploid inducer to create an F2-H haploid population. These F2-H populations were grown at three locations. Individual haploid plants were screened with daily pollen checks (HMF scores) and kernel number measurements as previously described. The observed phenotypes (see Table 3) were then compared against Axiom genotypic results for a population of 475 plants and QTL association mapping performed using the same method as had been performed for the F1-H generation. The QTLs previously identified on chromosomes 2 and 10 (Table 2) failed to score as significantly in this second generation. However, the chromosome 7 QTL was equally strong in the second generation (LOD score>5) in both the populations where it was expected. This chromosome 7 QTL is designated SCD-QTL1.

TABLE 3
All QTL observed in F2-H generation associated mapping studies sorted by LOD
score intensity. LOD scores of >5 are considered significant. The only
loci from these analyses that also appeared as significant QTL in the F1-H
populations occur on chromosome 7 between 119.8-133.4 Mb within SCD-QTL1.

| | | B73_v4 | B73_v4 | | | | |
|---|---|---|---|---|---|---|---|
| | | Physical | Physical | | | | Significant |
| F2-H | | Left | Right | Plants | | | in F1-H |
| Population | | Flanking | Flanking | in | LOD | Explained | Generation |
| Parents | Chromosome | Marker | Marker | Study | Score | Variance | Study |
| SYN004 - | 7 | 123,731,235 | 123,980,818 | 240 | 20.23 | 16.4 | Y |
| NP2222 | | | | | | | |
| SYN004 - | 7 | 119,807,228 | 121,417,971 | 240 | 16.77 | 14.13 | Y |
| NP2222 | | | | | | | |
| SYN004 - | 1 | 279,919,886 | 279,939,641 | 240 | 10.32 | 7.44 | N |
| NP2222 | | | | | | | |
| SYN004 - | 7 | 132,975,504 | 133,378,292 | 310 | 6.63 | 7.28 | Y |
| SYN006 | | | | | | | |
| SYN004 - | 3 | 208,702,590 | 208,734,331 | 310 | 6.15 | 5.87 | N |
| SYN006 | | | | | | | |
| SYN004 - | 2 | 219,293,152 | 219,456,766 | 240 | 5.74 | 4.03 | N |
| NP2222 | | | | | | | |
| SYN004 - | 2 | 53,485,817 | 53,591,523 | 310 | 4.89 | 4.55 | N |
| SYN006 | | | | | | | |
| SYN004 - | 1 | 52,592,197 | 53,184,649 | 240 | 4.76 | 3.26 | N |
| NP2222 | | | | | | | |
| SYN004 - | 9 | 108,418,716 | 110,030,821 | 310 | 4.32 | 4.62 | N |
| SYN006 | | | | | | | |
| NP2222 - | 6 | 127,319,447 | 128,895,525 | 196 | 2.97 | 6.51 | N |
| SYN007 | | | | | | | |
| NP2222 - | 6 | 129,108,045 | 131,093,258 | 196 | 2.88 | 6.31 | N |
| SYN007 | | | | | | | |
| SYN004 - | 7 | 177,902,979 | 178,663,798 | 310 | 2.63 | 2.78 | N |
| SYN006 | | | | | | | |
| SYN004 - | 7 | 173,494,985 | 173,859,507 | 310 | 2.35 | 2.5 | N |
| SYN006 | | | | | | | |
| SYN004 - | 1 | 286,170,579 | 286,208,468 | 310 | 2.11 | 2.23 | N |
| SYN006 | | | | | | | |

### Example 3. Fine-Mapping the Boundaries of the QTL

### Fine-Mapping and SNP Genotyping in F2-H Populations

In the same F2-H populations grown for QTL validation, plants were also genotyped within the expected QTL interval on chromosome 7 using SNP-based TaqMan assay markers with predicted polymorphisms between the parental alleles of the F2-H populations (see Table 4). These markers covered a region of chromosome 7 from 113.9-154.6 Mb (positions from B73_v4 reference genome) and were selected and/or designed based on genome sequences for the parental lines. Comparing these results, the genotype from the SCD-traited parent (Line B in Table 5) is associated with higher-than-average haploid pollen fertility rates between markers SM0077AQ and SM11142. These two markers flank a 2.54 Mb region (B73_v4 coordinates), containing 46 annotated gene models, and was 74% diagnostic of pollen fertility in this study. To confirm the statistical significance of the interval defined by recombination mapping, 466 haploids from the F2-H population that were either A-A or B-B genotypes at markers SM0077AQ and SM11142 were selected for a single-factor ANOVA analysis. Comparison of the HMF scores for the A Line group versus the B Line group (averages of 1.94 and 6.27 HMF score units respectively), were found to be statistically significantly different with a P-value equal to 8.44*10⁻³². Based on all analyses, we define SCD-QTL1 by the region of chromosome 7 flanked by markers SM0077AQ and SM11142, corresponding to their physical locations in B73 which was determined by searching the DNA sequences associated with each of the TaqMan assays in that reference genome. DNA sequences for these TaqMan markers are in Table 6.

TABLE 4
Locations of polymorphic SNP-assay markers used for fine-mapping
of QTL boundaries in F2-H populations. Markers were designed
and selected based on predicted differences between parents
of bi-allelic populations based on genome sequences, then validated
in parent materials. Not all markers were able to be validated
and so not all were included in the final analysis.

| Chromosome | Marker Name | B73_v4 Physical Position | Unit |
|---|---|---|---|
| 7 | SM4510 | 112,540,490 | bp |
| 7 | SM3802 | 112,555,427 | bp |
| 7 | SM1496EQ | 113,574,327 | bp |
| 7 | SM3773 | 114,658,138 | bp |
| 7 | SM3428 | 115,920,405 | bp |
| 7 | SM1157AQ | 118,721,983 | bp |
| 7 | SM11124 | 118,786,031 | bp |
| 7 | SM11129 | 119,353,581 | bp |
| 7 | SM11120 | 119,377,549 | bp |
| 7 | SM11108 | 119,380,216 | bp |
| 7 | SM11135 | 119,506,978 | bp |
| 7 | SM11123 | 119,602,354 | bp |
| 7 | SM11115 | 119,801,633 | bp |
| 7 | SM11130 | 120,115,075 | bp |
| 7 | SM11114 | 120,243,414 | bp |
| 7 | SM11117 | 120,391,868 | bp |
| 7 | SM11128 | 121,270,456 | bp |
| 7 | SM11137 | 121,355,533 | bp |
| 7 | SM11133 | 121,398,656 | bp |
| 7 | SM11121 | 121,546,326 | bp |
| 7 | SM11127 | 121,550,070 | bp |
| 7 | SM11109 | 121,654,842 | bp |
| 7 | SM11113 | 121,679,311 | bp |
| 7 | SM11126 | 121,736,796 | bp |
| 7 | SM11118 | 121,740,908 | bp |
| 7 | SM11112 | 121,986,702 | bp |
| 7 | SM11111 | 122,059,610 | bp |
| 7 | SM11122 | 122,206,231 | bp |
| 7 | SM11116 | 122,356,146 | bp |
| 7 | SM11136 | 122,713,813 | bp |
| 7 | SM11103 | 122,751,504 | bp |
| 7 | SM11105 | 122,819,769 | bp |
| 7 | SM11134 | 122,826,461 | bp |
| 7 | SM11138 | 122,834,646 | bp |
| 7 | SM11131 | 122,852,574 | bp |
| 7 | SM11119 | 123,259,873 | bp |
| 7 | SM11139 | 123,473,353 | bp |
| 7 | SM11104 | 123,636,820 | bp |
| 7 | SM0077AQ | 123,982,736 | bp |
| 7 | SM11165 | 124,122,936 | bp |
| 7 | SM11163 | 124,766,387 | bp |
| 7 | SM11164 | 126,034,078 | bp |
| 7 | SM11161 | 126,321,545 | bp |
| 7 | SM11142 | 126,520,521 | bp |
| 7 | SM11141 | 126,703,448 | bp |
| 7 | SM11143 | 126,939,888 | bp |
| 7 | SM11162 | 128,277,353 | bp |
| 7 | SM11107 | 128,285,939 | bp |
| 7 | SM11106 | 128,419,115 | bp |
| 7 | SM11132 | 128,495,322 | bp |
| 7 | SM11125 | 128,589,230 | bp |
| 7 | SM11140 | 128,750,237 | bp |
| 7 | SM11110 | 128,989,703 | bp |
| 7 | SM4152 | 129,166,619 | bp |
| 7 | SM11101 | 130,286,707 | bp |
| 7 | SM11100 | 130,287,037 | bp |
| 7 | SM1255AQ | 131,676,810 | bp |
| 7 | SM1255CQ | 131,676,828 | bp |
| 7 | SM1255BQ | 131,676,933 | bp |
| 7 | SM11102 | 134,895,500 | bp |
| 7 | SM3975 | 135,238,418 | bp |
| 7 | SM2485 | 139,327,889 | bp |
| 7 | SM0711BQ | 139,470,524 | bp |
| 7 | SM0711AQ | 139,470,715 | bp |
| 7 | SM4519 | 139,492,603 | bp |
| 7 | SM3432 | 154,611,128 | bp |

TABLE 5
Recombination groups observed in segregating F2-H haploid population from 123.4-128.8
Mb using polymorphic TaqMan assays. Genotypes were scored as matching either Parent
A (no SCD trait) or Parent B (with SCD trait) at each marker and then compared against
HMF pollen scores. Genotypic groups in the region were compiled, and the HMF scores
for plants matching that genotype averaged to get a measure of SCD performance based
on genotype in this region. Based on recombination locations (switches from A to B
or vice-versa) and the average HMF scores, QTL-SCD1 lies between markers SM0077AQ
and SM11142. Average HMF for all haploids in the F2-H population was 4.0.

| | | | Physical Position | | |
|---|---|---|---|---|---|
| | | | 123,473,353 | 123,636,820 | 123,982,736 |
| | | | Marker Name | | |
| | | | SM11139 | SM11104 | SM0077AQ |
| | Number | | Genotype at | Genotype at | Genotype at |
| | of Plants | Avg | Marker | Marker | Marker |
| | with this | HMF | Position | Position | Position |
| Group | Genotype | Score | (A or B) | (A or B) | (A or B) |
| NP2222 Diploid | 14 | 13 | A | A | A |
| Parent (A Line) | | | | | |
| SYN004 Diploid | 11 | 13 | B | B | B |
| Parent (B Line) | | | | | |
| F2-H Haploids | 199 | 6 | B | B | B |
| F2-H Haploids | 12 | 6 | B | B | B |
| F2-H Haploids | 4 | 5 | B | B | B |
| F2-H Haploids | 2 | 0 | B | B | A |
| F2-H Haploids | 5 | 6 | A | A | A |
| F2-H Haploids | 6 | 2 | A | A | A |
| F2-H Haploids | 247 | 2 | A | A | A |

| | | | Physical Position | | | |
|---|---|---|---|---|---|---|
| | | | 126,520,521 | 128,419,115 | 128,589,230 | 128,750,237 |
| | | | Marker Name | | | |
| | | | SM11142 | SM11106 | SM11125 | SM11140 |
| | Number | | Genotype at | Genotype at | Genotype at | Genotype at |
| | of Plants | Avg | Marker | Marker | Marker | Marker |
| | with this | HMF | Position | Position | Position | Position |
| Group | Genotype | Score | (A or B) | (A or B) | (A or B) | (A or B) |
| NP2222 Diploid | 14 | 13 | A | A | A | A |
| Parent (A Line) | | | | | | |
| SYN004 Diploid | 11 | 13 | B | B | B | B |
| Parent (B Line) | | | | | | |
| F2-H Haploids | 199 | 6 | B | B | B | B |
| F2-H Haploids | 12 | 6 | B | A | A | A |
| F2-H Haploids | 4 | 5 | A | A | A | A |
| F2-H Haploids | 2 | 0 | A | A | A | A |
| F2-H Haploids | 5 | 6 | B | B | B | B |
| F2-H Haploids | 6 | 2 | A | B | B | B |
| F2-H Haploids | 247 | 2 | A | A | A | A |

TABLE 6
DNA sequences for markers defining SCD-QTL1

| Marker Name | SM0077AQ | SM11142 |
|---|---|---|
| B73_v4 Physical Position | 123,982,736 | 126,520,521 |
| Forward Primer | SEQ ID NO: 1 | SEQ ID NOs: 3 |
| | | and 4 |
| Reverse Primer | SEQ ID NO: 2 | SEQ ID NO: 5 |
| Target Sequence | SEQ ID NO: 6 | SEQ ID NO: 7 |

### Example 4. Comparison Mapping of SCD-QTL1 to Previously Published QTL and/or SNPs

In two published genome-wide association mapping studies, Ma (2018) and Yang (2019) previously identified QTL associated with SCD on maize chromosome 7. However, the QTL defined in these studies do not overlay SCD-QTL1 as defined in this document using the B73_v4 reference genome. Ma (2018) identified two SNPs associated with SCD in their mapping population, one at 113,165,535 and one at 170,671,328 bp in the B73_v2 genome (available at maizegdb.org). A BLAST search of the target sequence (SEQ ID NO: 6) for marker SM0077AQ at maizegdb.org shows the relative location of that sequence between B73_v2 and B73_v4 places the left boundary of SCD-QT11 starting at 120,202,757 bp in B73_v2. This region does not overlap with that identified by Ma (2018). Yang (2019) identified 3 QTL associated with chromosome 7 in two different maize lines and provided their approximate genomic locations in cM (map distance) in a linkage map derived for their study that they describe as agreeing with maps derived for B73_v2. They also provided chromosome bin numbers for the QTL, but these are imprecise as bins can be very large (bin 7.02, for example, spans from 13.8-128.1 Mb covering 62% of chromosome 7 in the B73_v2 reference). Using the study linkage map, the three identified loci (qHMF7a, qHMF7b, and qHMF7c) are located at 26.51-28.51 cM, 41.51-43.51 cM, and 74.41 cM. SCD-QTL1, according to the target sequence BLAST results in the maizeGDB genome browser is more than 97.2 cM from the start of the chromosome, and thus cannot overlap with any of the QTL identified in Yang (2019). Having reviewed these relative coordinates, we are confident that SCD-QTL1 represents a new QTL not previously identified in the academic literature.

### Example 5. Phenotypic Analysis

To compare phenotypes of spontaneous chromosome doubling (SCD) observed in the high-doubling rate lines observed in the original screen, two lines, SYN007 and SYN004 were selected for more in-depth phenotypic analysis including pollen viability testing and ploidy analysis of various tissue types. Ploidy analysis can easily be done on a variety of plant tissues using flow cytometry to determine average DNA content of a population of nuclei extracted from fresh tissue (Dolezel, 2007). No SCD was observed in diploids from these lines when seedling leaf, adult leaf, pollen, and husk tissues were analyzed using flow cytometry. However, in haploid plants, doubling was readily detectable using flow cytometry from male reproductive tissues (anthers, pollen, spikelets). However, SCD was not observed in the vegetative tissues (leaf, root, stem), indicating that developmentally, the SCD observed in these lines occurs only after initiation of the floral organs.

Pollen metabolic activity can be determined using a 3-(4,5-dimethylthiazol-2-yl)-2,5-diphenyltetrazolium bromide (MTT) colorimetric assay (Firmage and Dafni, 2001). In the presence of active oxidoreductase enzymes within the pollen grains, MTT is reduced from a yellow solution to an insoluble purple formazan. Pollen grains that turn purple in the presence of MTT are considered metabolically active and considered viable. Because haploid pollen is always sterile in the absence of chromosome doubling, the presence of viable pollen in a haploid plant is a direct result of SCD. In this experiment, anthers were collected from each of the two lines at the stage of mature pollen shed. Then, pollen from individual anthers was released into the MTT solution by gently grinding the tissue with a plastic pestle. Pollen collected from individual anthers of line SYN007 typically showed a mixture of viable and non-viable pollen. However, pollen from individual anthers of line SYN004 was either 100% viable or 0% viable. Furthermore, when individual anthers were compared by length, anthers with viable (i.e. doubled) pollen were twice the length of anthers with non-viable (i.e. not doubled) pollen in line SYN004 only. Anthers within florets were typically all the same length. This data implies that the developmental timing of SYN007 SCD is much later, after the progenitor cells of the pollen are already specified and distinct from one another. However, SYN004 SCD must happen at earlier stages of floral development at roughly the time that individual florets are being specified, but before the anthers begin to form. These conclusions are based on principles of maize floral development, where initially a small group of meristematic cells are the founders of the entire tassel through a series of differentiation events. If the SCD happens very early in development, we expect large portions, maybe even the whole tassel, to demonstrate SCD. The phenotype of the enlarged anthers (larger cells are indicative of higher ploidy level) in SYN004, and entire populations of viable pollen, implies that the SCD in that line happens in a cell that precedes the entire anther in the developmental progression. The mixture of pollen viability within individual anthers, and the absence of entirely doubled (larger) anthers, implies the SGD in SYN007 happens later, after most of the floral organs are already present, but before the pollen is done maturing.

### Example 6. QTL Penetrance in Diverse Germplasm and in the Absence of Additional Doubling Treatment

It is common for QTL penetrance to be germplasm dependent based on other, often unidentified genetic factors segregating in diverse germplasm backgrounds. To test how widely SCD-QTL1 would penetrate in a more diverse set of lines, a panel of 17 elite inbred lines were selected from among Syngenta's North America breeding populations. These lines represented several heterotic groups (stiff stalk, non-stiff stalk, and iodent) and a range of maturity groups. To generate our diverse germplasm populations for phenotypic screening, SYN004, the QTL donor, was crossed by each of the 17 elites. Those F1 populations were then backcrossed to the 17 recurrent parents to generate a BC1 generation and backcrossed once more to generate a BC2 generation. In the BC2 populations, SNP assay markers defining the QTL (Table 5) were used to genotype lines across the QTL interval and identify plants that were heterozygous for the favorable (SYN004, doubling) allele. Those heterozygotes were then crossed by a haploid inducer line and resulting haploids were identified in mature seed based on a R-nj color maker. From the resulting haploids, 50% of them were positive for the favorable allele, and 50% were negative, creating an ideal control group to compare performance in the field.

The mature haploid seed was direct seeded into a field, and seedlings were again genotyped with SNP markers (Table 5) to confirm QTL allele status as well as to remove any potential diploids from the population. Plants were grown under standard field nursery conditions with the addition of a shade-cloth cover to reduce light intensity until pollen shed. Individual plant phenotypes were collected for each haploid measuring the number of days of pollen shed and the number of kernels per ear after a self-pollination. In the absence of a colchicine or other chemical doubling treatment, the expectation for the haploid phenotypes is zero days of pollen shed and zero kernels. Indeed, this expectation was observed in the recurrent parent controls where no favorable allele of SCD-QTL1 was present. However, in the BC2-haploid population, many lines were observed to have more fertile pollen present in individuals with the SCD-QTL1 favorable allele genotype (Table 7). This was true in 7 of the 17 elite lines, indicating a moderate level of QTL penetrance. These same lines had a modest seed set (5 of 17 lines had more kernels per ear in the presence of the SCD1-QTL favorable allele), perhaps indicating that an additional factor contributing to doubling in the female (haploid female fertility) may by absent in some lines.

TABLE 7
Pollen and kernel phenotypes in BC2-Haploid populations
grown in the absence of chemical doubling treatment

| | | | Average | | |
|---|---|---|---|---|---|
| | | | Number | | Average |
| | | | of Days | Percent | Number |
| | | Percent | of | of Selfed | of |
| BC2 Recurrent | | of Plants | Pollen | Plants | Kernels |
| Parent Inbred | | that Shed | per | with | per |
| Line | SCD1-QTL Genotype | Pollen | Plant | Kernels | Plant |
| Line 1 | Unfavorable Allele | 22.2% | 1.2 | 5.6% | 1.3 |
| Line 1 | SCD Allele | 42.1% | 2.5 | 10.5% | 2.0 |
| Line 2 | Unfavorable Allele | No Data | No Data | No Data | No Data |
| Line 2 | SCD Allele | 1.8% | 0.1 | 1.8% | 0.1 |
| Line 3 | Unfavorable Allele | 12.1% | 0.5 | 0.0% | 0.0 |
| Line 3 | SCD Allele | 75.9% | 4.2 | 17.9% | 0.6 |
| Line 4 | Unfavorable Allele | 32.0% | 2.1 | 20.0% | 3.1 |
| Line 4 | SCD Allele | 25.0% | 1.4 | 6.3% | 1.7 |
| Line 5 | Unfavorable Allele | 27.3% | 1.5 | 0.0% | 0.0 |
| Line 5 | SCD Allele | 15.0% | 1.0 | 5.0% | 0.4 |
| Line 6 | Unfavorable Allele | 3.4% | 0.2 | 0.0% | 0.0 |
| Line 6 | SCD Allele | 8.7% | 0.6 | 0.0% | 0.0 |
| Line 7 | Unfavorable Allele | 0.0% | 0.0 | 0.0% | 0.0 |
| Line 7 | SCD Allele | 0.0% | 0.0 | 0.0% | 0.0 |
| Line 8 | Unfavorable Allele | 71.9% | 5.8 | 46.9% | 2.6 |
| Line 8 | SCD Allele | 90.6% | 6.5 | 50.0% | 5.3 |
| Line 9 | Unfavorable Allele | 14.3% | 0.6 | 0.0% | 0.0 |
| Line 9 | SCD Allele | 5.6% | 0.2 | 0.0% | 0.0 |
| Line 10 | Unfavorable Allele | 81.4% | 6.1 | 51.2% | 9.6 |
| Line 10 | SCD Allele | 94.3% | 6.5 | 51.4% | 11.0 |
| Line 11 | Unfavorable Allele | 78.6% | 4.4 | 2.4% | 0.0 |
| Line 11 | SCD Allele | 64.3% | 3.6 | 7.1% | 0.1 |
| Line 12 | Unfavorable Allele | 52.0% | 3.0 | 36.0% | 6.3 |
| Line 12 | SCD Allele | 77.8% | 4.6 | 66.7% | 10.1 |
| Line 13 | Unfavorable Allele | 30.0% | 1.8 | 22.5% | 1.5 |
| Line 13 | SCD Allele | 50.0% | 3.6 | 40.0% | 5.6 |
| Line 14 | Unfavorable Allele | 0.0% | 0.0 | 0.0% | 0.0 |
| Line 14 | SCD Allele | 0.0% | 0.0 | 0.0% | 0.0 |
| Line 15 | Unfavorable Allele | 29.4% | 1.2 | 0.0% | 0.0 |
| Line 15 | SCD Allele | 50.0% | 3.6 | 5.0% | 0.1 |
| Line 16 | Unfavorable Allele | 78.8% | 5.0 | 18.2% | 2.2 |
| Line 16 | SCD Allele | 77.4% | 5.0 | 29.0% | 4.8 |
| Line 17 | Unfavorable Allele | 8.7% | 0.4 | 4.3% | 0.0 |
| Line 17 | SCD Allele | 10.5% | 0.6 | 5.3% | 0.1 |
| Diploid Recurrent | Unfavorable Allele | 100.0% | 9.5 | 100.0% | 320.3 |
| Parent | | | | | |
| Diploid SCD | SCD Allele | 100.0% | 9.0 | 100.0% | 318.7 |
| Donor Parent | | | | | |

### Example 7. QTL Penetrance in Diverse Germplasm in Combination with Colchicine Doubling Treatment

To test whether the SCD-QTL1 could be used in combination with a chemical doubling treatment, such as colchicine, and have an additive effect on haploid male fertility, we took the 17 elite BC2 lines from Example 6 through one additional backcross generation to produce a BC3. Genotyping was used to identify heterozygotes at SCD-QTL1, and those materials were then crossed by a haploid inducer, embryos isolated from the immature ears, and haploids regenerated for transplant into the field. Immediately after isolation, embryos from each of the 17 BC3 populations were split into 2 treatment groups. One group received a colchicine doubling treatment, and the second group did not receive any chemical doubling agent. Thus, there were 4 treatment groups to compare within each elite BC3 population: (1) SCD-QTL1 with colchicine (haploid male fertility plus colchicine), (2) unfavorable QTL allele with colchicine (positive control), (3) SCD-QTL1 without colchicine (haploid male fertility alone), (4) and unfavorable QTL allele without colchicine (negative control). All lines were planted across three replications of the trial over the course of approximately 6 months, and the data was pooled for analysis.

In this trial, sample sizes were large enough (minimum of 50 haploids per treatment group) to enable statistical analysis to confirm whether observed differences in kernel set between groups with and without the favorable allele of SCD-QTL1 were statistically significant using a Student's T-Test (Table 8). Three of the 17 lines had a significant increase (p-value<0.05) in kernel set in the presence of SCD-QTL1 while 3 of the 17 lines showed a significant decrease (p-value<0.05). Seven of the lines did not show any significant impact of the QTL, and 4 were recalcitrant to all double-haploid procedures (these were excluded from the analysis). Amongst the 7 lines that did not show any statistically significant differences, several lines had positive but non-significant trends. The 3 lines that showed a negative association with the SCD-QTL1 likely indicate another factor co-segregating with SCD-QTL1, which is still a relatively large region (˜2 Mb) and contains 62 potential gene candidates. In double-haploid production, a key metric for success is the number of individual haploids that produce ears with greater than 5 kernels per ear with a colchicine treatment. Comparing only the colchicine-treated groups (which performed overall better in both pollen and kernel production than the SCD-QTL1 alone), 5 lines showed an increase in the percent of haploids that produced a selfed ear with greater than 5 kernels, indicating that in some lines, the presence of the SCD-QTL1 favorable allele would increase the efficiency of double-haploid production (Table 8).

TABLE 8
Pollen and kernel phenotypes for the
4 treatment groups in Example 7

| | | | Average | | |
|---|---|---|---|---|---|
| | | | Number | Average | Percent |
| BC3 | | | of Days | Number | of |
| Recurrent | | | of | of | Plants |
| Parent | | | Pollen | Kernels | with |
| Inbred | SCD1-QTL | Chemical | per | per | Ears >5 |
| Line | Genotype | Treatment | Plant | Plant | kernels |
| Line 1 | SCD Allele | Colchicine | 3.1 | 23.8 | 53.9% |
| Line 1 | Unfavorable | Colchicine | 2.4 | 13.5 | 26.6% |
| | Allele | | | | |
| Line 1 | SCD Allele | None | 0.66 | 1.05 | 3.6% |
| Line 1 | Unfavorable | None | 0.57 | 0.72 | 4.3% |
| | Allele | | | | |
| Line 2 | SCD Allele | Colchicine | 1.72 | 5 | 12.0% |
| Line 2 | Unfavorable | Colchicine | 2.35 | 9.62 | 13.5% |
| | Allele | | | | |
| Line 2 | SCD Allele | None | 0.03 | 0.166 | 0.0% |
| Line 2 | Unfavorable | None | 0.18 | 6.79 | 3.1% |
| | Allele | | | | |
| Line 3 | SCD Allele | Colchicine | 1.76 | 7.62 | 6.9% |
| Line 3 | Unfavorable | Colchicine | 1.45 | 9.03 | 9.1% |
| | Allele | | | | |
| Line 3 | SCD Allele | None | 0.75 | 0.11 | 1.2% |
| Line 3 | Unfavorable | None | 0.21 | 0 | 0.0% |
| | Allele | | | | |
| Line 4 | SCD Allele | Colchicine | 0.04 | 0 | 0.0% |
| Line 4 | Unfavorable | Colchicine | 0.57 | 7.1 | 13.2% |
| | Allele | | | | |
| Line 4 | SCD Allele | None | 0 | 0 | 0.0% |
| Line 4 | Unfavorable | None | 0.55 | 23.8 | 10.2% |
| | Allele | | | | |
| Line 5 | SCD Allele | Colchicine | 2.34 | 2.31 | 3.1% |
| Line 5 | Unfavorable | Colchicine | 2.19 | 4.88 | 7.6% |
| | Allele | | | | |
| Line 5 | SCD Allele | None | 0.04 | 0 | 0.0% |
| Line 5 | Unfavorable | None | 0.28 | 10.5 | 4.8% |
| | Allele | | | | |
| Line 6 | SCD Allele | Colchicine | 1.675 | 11.23 | 25.0% |
| Line 6 | Unfavorable | Colchicine | 2.01 | 19.09 | 27.2% |
| | Allele | | | | |
| Line 6 | SCD Allele | None | 0.03 | 0.006 | 0.0% |
| Line 6 | Unfavorable | None | 0.2 | 7.75 | 4.1% |
| | Allele | | | | |
| Line 7 | SCD Allele | Colchicine | 1.58 | 53.5 | 37.9% |
| Line 7 | Unfavorable | Colchicine | 1.298 | 54.18 | 29.9% |
| | Allele | | | | |
| Line 7 | SCD Allele | None | 0.055 | 0 | 0.0% |
| Line 7 | Unfavorable | None | 0.026 | 0.23 | 0.7% |
| | Allele | | | | |
| Line 8 | SCD Allele | Colchicine | 1.72 | 37.34 | 31.1% |
| Line 8 | Unfavorable | Colchicine | 1.466 | 26.17 | 32.0% |
| | Allele | | | | |
| Line 8 | SCD Allele | None | 0.605 | 1.85 | 7.9% |
| Line 8 | Unfavorable | None | 1.034 | 50.21 | 7.0% |
| | Allele | | | | |
| Line 9 | SCD Allele | Colchicine | 2.58 | 27.9 | 36.0% |
| Line 9 | Unfavorable | Colchicine | 2.51 | 24.067 | 29.3% |
| | Allele | | | | |
| Line 9 | SCD Allele | None | 0.35 | 3.12 | 5.2% |
| Line 9 | Unfavorable | None | 0.13 | 2.68 | 1.2% |
| | Allele | | | | |
| Line 10 | SCD Allele | Colchicine | 1.12 | 23.17 | 34.2% |
| Line 10 | Unfavorable | Colchicine | 1.005 | 13.75 | 26.4% |
| | Allele | | | | |
| Line 10 | SCD Allele | None | 0.82 | 2.66 | 15.9% |
| Line 10 | Unfavorable | None | 0.75 | 2.275 | 10.7% |
| | Allele | | | | |
| Line 11 | SCD Allele | Colchicine | 1.09 | 4.97 | 8.7% |
| Line 11 | Unfavorable | Colchicine | 1.2 | 18.11 | 17.5% |
| | Allele | | | | |
| Line 11 | SCD Allele | None | 0.22 | 2.07 | 4.9% |
| Line 11 | Unfavorable | None | 0.41 | 7.02 | 5.7% |
| | Allele | | | | |
| Line 12 | SCD Allele | Colchicine | 1.25 | 19.56 | 28.5% |
| Line 12 | Unfavorable | Colchicine | 0.96 | 22.12 | 21.3% |
| | Allele | | | | |
| Line 12 | SCD Allele | None | 1.13 | 8.79 | 18.2% |
| Line 12 | Unfavorable | None | 0.72 | 11.49 | 11.0% |
| | Allele | | | | |
| Line 13 | SCD Allele | Colchicine | 1.45 | 32.5 | 25.8% |
| Line 13 | Unfavorable | Colchicine | 1.33 | 24.5 | 24.7% |
| | Allele | | | | |
| Line 13 | SCD Allele | None | 2.14 | 3.72 | 20.7% |
| Line 13 | Unfavorable | None | 1 | 1.36 | 6.5% |
| | Allele | | | | |
| Line 14 | SCD Allele | Colchicine | 1.28 | 31 | 29.9% |
| Line 14 | Unfavorable | Colchicine | 1.1 | 40.57 | 26.2% |
| | Allele | | | | |
| Line 14 | SCD Allele | None | 0.04 | 2.24 | 1.7% |
| Line 14 | Unfavorable | None | 0.06 | 0.03 | 0.0% |
| | Allele | | | | |
| Line 15 | SCD Allele | Colchicine | 2.66 | 45.47 | 64.9% |
| Line 15 | Unfavorable | Colchicine | 2.81 | 72.9 | 72.2% |
| | Allele | | | | |
| Line 15 | SCD Allele | None | 0.21 | 6.46 | 8.0% |
| Line 15 | Unfavorable | None | 0.36 | 9.34 | 11.0% |
| | Allele | | | | |
| Line 16 | SCD Allele | Colchicine | 1.65 | 31.56 | 34.1% |
| Line 16 | Unfavorable | Colchicine | 1.67 | 28.03 | 37.6% |
| | Allele | | | | |
| Line 16 | SCD Allele | None | 0.97 | 6.67 | 21.9% |
| Line 16 | Unfavorable | None | 0.85 | 5.3 | 15.0% |
| | Allele | | | | |
| Line 17 | SCD Allele | Colchicine | 2.63 | 52.86 | 49.1% |
| Line 17 | Unfavorable | Colchicine | 2.51 | 58.48 | 50.4% |
| | Allele | | | | |
| Line 17 | SCD Allele | None | 1.23 | 2.41 | 1.6% |
| Line 17 | Unfavorable | None | 0.83 | 0.29 | 0.7% |
| | Allele | | | | |

1. A method of selecting a first maize plant or germplasm that displays spontaneous chromosome doubling (“SCD”) when in a haploid state, the method comprising:
a. isolating nucleic acids from the first maize plant or germplasm;
b. detecting in the first maize plant or germplasm SCD-QTL1 comprising SEQ ID NOs: 6 and 7 that is associated with spontaneous chromosome doubling; and
c. selecting the first maize plant or germplasm, or selecting a progeny of the first maize plant or germplasm, comprising SCD-QTL1 that is associated with spontaneous chromosome doubling.

2. The method of claim 1, further comprising crossing said selected first maize plant or germplasm with a second maize plant or germplasm to produce a progeny maize plant, wherein the progeny maize plant or germplasm displays increased spontaneous chromosome doubling (“SCD”) when in a haploid state.

3. The method of claim 1, wherein the SCD-QTL1 is detected using a composition comprising a detectable label.

4. A method of obtaining a spontaneously doubled haploid plant, the method comprising:
a. obtaining a first maize plant comprising SCD-QTL1 comprising SEQ ID NOs: 6 and 7 that is associated with spontaneous chromosome doubling;
b. crossing the first maize plant with a second maize plant;
c. obtaining progeny from the cross of step b and selecting haploid progeny therefrom; and
d. allowing the haploid progeny to undergo spontaneous chromosome doubling; thereby obtaining a spontaneously doubled haploid plant.

5. The method of claim 3, wherein the spontaneously doubled haploid plant comprises reproductive tissues competent to produce fertile gametes.

6. The method of claim 4, wherein the spontaneously doubled haploid plant is male fertile.

7. The method of claim 3, wherein the first maize plant is a haploid inducer maize plant.

8. The method of claim 6, wherein the first maize plant comprises a mutation in CENH3 or IG1.

9. The method of claim 3, wherein the second maize plant is a haploid inducer maize plant.

10. The method of claim 8, wherein the second maize plant comprises a mutation in ZmMATL.

11. A method of introgressing a locus of interest, the method comprising:
a. obtaining a first maize plant comprising SCD-QTL1 comprising SEQ ID NOs: 6 and 7 that is associated with spontaneous chromosome doubling;
b. crossing the first maize plant with a second maize plant comprising the locus of interest;
c. obtaining progeny from the cross of step b.;
d. optionally backcrossing the progeny of step c. with the first maize plant or the second maize plant;
e. screening the progeny of step c. or step d. for presence of the locus of interest and the SCD QTL; and
f. selecting a progeny at least heterozygous for the locus of interest and at least heterozygous for the SCD QTL;
thereby obtaining a maize plant comprising the locus of interest and the SCD QTL.

12. The method of claim 10, wherein screening the progeny for presence of the locus of interest and the SCD-QTL is screened by genotype.

13. The method of claim 10, wherein screening the progeny for presence of the locus of interest and the SCD-QTL is screened by phenotype.

14. The method of claim 10, wherein the locus of interest comprises a quantitative trait locus that is associated with a trait selected from the group consisting of increased yield, increased water optimization, increased disease resistance, increased drought tolerance, and increased herbicide resistance.

15. The method of claim 13, wherein the locus of interest comprises a transgene.

16. A maize plant produced by the method of claim 10.

17. The maize plant of claim 15, wherein the maize plant belongs to the non-stiff stalk heterotic group, the stiff stalk heterotic group, or the tropical heterotic group.

18. The maize plant of claim 15, wherein the maize plant is a hybrid maize plant or an elite maize plant.

19. A maize progeny plant produced from the maize plant of claim 15.

20. A detection kit comprising SEQ ID NO: 1, SEQ ID NO: 2, SEQ ID NO: 3, SEQ ID NO: 4, and SEQ ID NO: 5.

21. (canceled)


SEQUENCE LISTING (7 sequences)

SEQ ID NO: 1 - DNA, 17 bp, synthetic construct
  misc_feature 1..17: note = Forward primer
  source 1..17: mol_type = other DNA
cgtcgggctgcactgat

SEQ ID NO: 2 - DNA, 16 bp, synthetic construct
  misc_feature 1..16: note = reverse primer
  source 1..16: mol_type = other DNA
gcagcaccccgatcct

SEQ ID NO: 3 - DNA, 13 bp, synthetic construct
  misc_feature 1..13: note = forward primer
  source 1..13: mol_type = other DNA
ccgggccacccag

SEQ ID NO: 4 - DNA, 20 bp, synthetic construct
  misc_feature 1..20: note = reverse primer
  source 1..20: mol_type = other DNA
cggcagaggcagatctacac

SEQ ID NO: 5 - DNA, 24 bp, synthetic construct
  misc_feature 1..24: note = reverse primer for marker SM11142
  source 1..24: mol_type = other DNA
atgccctgaactaggactactaaa

SEQ ID NO: 6 - DNA, 650 bp, Zea mays
  variation 380: note = r is a or g
  variation 576: note = n is a, c, g, or t
  variation 578: note = n is a, c, g, or t
  variation 595: note = n is a, c, g, or t
  variation 620: note = n is a, c, g, or t
  source 1..650: mol_type = genomic DNA
tcgctcagttttgttgcgctctacattattactcaatcatcgccgctaaatgactcaactagctagctagccagcggctatactacagcacatagtttcttcagtgggggacaccgccggcagcagcgtaggggcatggagtcggctgctgcaggccgcacatcgccgtcagttgctgcgctatctgcgccgccaacagccccgcgacctggccgctttgcggctgctgctgcaggatggactggaggaccaagccgaagatcgcctggtaccggtgctgcggctccacctgcctgagctgctggcaacactgctgccgcaacgactggcactgaggcgagcagtagggcgtcgccgtcgggctgcactgatgcctcagraactcgacgcactggcccaggatcggggtgctgccaacgccgcaggttccctgcagctggcacgggcttggatgcggctgttggcacgggcatgggtgtggctggggatgaggctgaggccggggcggttgagtagggtagtggcatggtggcggcggcagatgaaccggcggcggcacatggaccggcggtggcngntggaccggcggcggcangtggaccggcggtgggagatgcacnggaggtggcaggtgaaccggaggtggcaga

SEQ ID NO: 7 - DNA, 1002 bp, Zea mays
  variation 52: note = n is a, c, g, or t
  variation 89: note = n is a, c, g, or t
  variation 143: note = n is a, c, g, or t
  variation 222: note = n is a, c, g, or t
  variation 502: note = r is a or g
  variation 598: note = n is a, c, g, or t
  variation 633: note = n is a, c, g, or t
  variation 648: note = n is a, c, g, or t
  variation 684: note = n is a, c, g, or t
  variation 695: note = n is a, c, g, or t
  variation 730..743: note = n is a, c, g, or t
  variation 761: note = n is a, c, g, or t
  variation 775: note = n is a, c, g, or t
  variation 785: note = n is a, c, g, or t
  variation 788: note = n is a, c, g, or t
  variation 791: note = n is a, c, g, or t
  variation 844: note = n is a, c, g, or t
  variation 861: note = n is a, c, g, or t
  variation 935: note = n is a, c, g, or t
  variation 939: note = n is a, c, g, or t
  variation 945: note = n is a, c, g, or t
  variation 952: note = n is a, c, g, or t
  variation 967: note = n is a, c, g, or t
  variation 998: note = n is a, c, g, or t
  source 1..1002: mol_type = genomic DNA
gtatgtgcttaagtttataaatttcaggtttcacctatccaccccctctaangcgactttcacgttgttgccacccctgtgcctaggcnaggctaggtgctagccagccctccccttcctccctttgtggcataggcagggcnccctcatgctagacctggttgggcctcaggctttgttgcgaccccacacgttgcactgggacctacagcgctgccactngtccatgcacattcccctgccgctagtgtgggtgtcgtcctacttcatgttgagctggactgtacactacctaccccaagcatgtggatctctcccgtctggtctctcggtagtagatccacagctgcccctctctaccgtgccgcatcgaccgatcctttgtagcccaacatcgtcactgcccagtctatgcttactgccatcgaaactgtgagctataataataagaactcacggttggaccatgatatcatcggcagaggcagatctacaccccggrccacccagtccgtggctcggagtataatccatgtgattccatataaatatgtatttaatctagtttaaaatagttatgataaaaggaggagaaaanttttagtagtcctagttcagggcataaaaaacttntatatctgcctctgntagtcggaatcgttgctactacatggtcaaaaaggnaaatgcatcgnatgtttctttcgaatgaaaagatgtaaggtgttannnnnnnnnnnnnngtgtcacattggaggacntaggcattgttcgntcattcctanccnatntggattgaatgggattagaaaaattatgaacgattttgacttgttcgagattnaaacccacgcaatcggnaattcacatggattgagaccaaaacgaacaaactcgactttatttggatactttagtattgatttcaatccatntatnttaagntggattntgatataaattacantctaacccacctcaacacatgtgtattgagntcga